(19) United States
(12) Patent Application Publication
(10) Pub. No.: US 2023/0233726 A1
(43) Pub. Date: Jul. 27, 2023

(54) A PORTABLE AIR TREATMENT SYSTEM AND A METHOD OF USING SAID AIR TREATMENT SYSTEM

(71) Applicant: INFUSER IP APS, Copenhagen N (DK)

(72) Inventors: Thomas Rosenørn, Birkerød (DK); Jonas Ingemar, København N (DK)

(21) Appl. No.: 18/001,348

(22) PCT Filed: Jun. 9, 2021
(86) PCT No.: PCT/EP2021/065496

§ 371 Date: Dec. 9, 2022

(30) Foreign Application Priority Data
Jun. 10, 2020 (DK) PA 2020 70364

(51) Int. Cl. A61L 9/015; A61L 9/20

(52) U.S. Cl. CPC A61L 9/015; A61L 9/20; A61L 2209/12; A61L 2209/14; A61L 2209/212; A61L 2209/111

(57) ABSTRACT

The present invention relates to an air treatment system (1) comprising a sterilization unit (2) arranged for producing ozone, a photooxidation unit (3) arranged for subjecting an air flow to a photooxidation process, and a control unit (4) arranged for in a first operational mode directing an air flow through the sterilization unit (2), and in a second operational mode directing an air flow through the photooxidation unit (3). By providing an air treatment system (1) which can operate in two different modes i.e. in a photooxidation or a sterilization mode, it is possible to specifically remove the undesirable pollutants in the air, e.g. removing either gas-phase

### CROSS REFERENCE TO RELATED APPLICATIONS

This application is a 371 filing of International Patent Application PCT/EP2021/065496 filed Jun. 9, 2021, which claims the benefit of priority to Danish patent application no. PA 2020 70364 filed Jun. 10, 2020, the disclosures of each of which is hereby incorporated by reference herein in its entirety.

### TECHNICAL FIELD

The present invention relates to an air treatment system, and a method of using said air treatment system.

### BACKGROUND

It is a well-known problem that air in different facilities such as homes, offices or in an industrial production rooms are contaminated with undesirable compounds and/or pollutants, e.g. volatile organic compounds (VOCs), allergens and infectious agents that affects the indoor air quality and accordingly the comfort and health of the occupants in said facility.

In this respect air treatment systems utilizing UV-radiation and/or ozone has proven highly advantageously. Using this technique it is possible to sterilize the air, and attain decomposition of organic compounds, e.g. VOCs at the same time.

Such air treatment systems are e.g. known from WO 97/34682 and WO 99/13956 in which air may be sterilized by exposing the air to UV-radiation, and organic compounds can be removed with ozone e.g. created during the UV-radiation. UV-light and ozone may be produced from the same UV-lamp, as the lamps can be arranged for emitting different wavelengths. From said patent applications, it is also known to control and regulate the ozone concentration that is emitted into the surroundings.

However, one of the main problems which these systems is that the systems are very complicated and the reaction times extremely long. Furthermore, a large numbers of UV-lamp must be applied in order to treat the air passing through the systems. This results in expensive systems, not only in equipment and installation but also in maintenance and operation.

Another problem is that the known air treatment systems often has been optimized to deal with one or more specific kinds of pollutants, e.g. VOCs having a specific origin, and even though other pollutants may be affected by the treatment, this is often not sufficient for eliminating or reducing the concentration of these other pollutants.

It is well known that infections spread when infectious agents are transmitted from an infected person to a susceptible person e.g. through contact, sprays and splashes and inhalation. Thus, if an infected person cough, talk, or sneeze into the air, this will create droplets which carry the infectious agent through the air, where it either may infect a person directly or land on a surface where is later indirectly can infect a person. It is therefore essential that both the air and surfaces in a giving room, e.g. a room in a hospital is sterilized/disinfected in order to prevent an infection to spread. However, even though the conventional air treatment systems to some extend can sterilize the air; they are not capable of producing the sufficient high concentration of ozone from ambient air to effectively sterilize contaminated surfaces in a room.

Thus, presently there does not exist a system which is optimized for both removing undesired pollutants and for sterilizing large volumes of indoor air and surfaces in a fast, simple and inexpensive manner. Accordingly, there exist demands for methods and systems that in an efficient and inexpensive way effectively can reduce hazardous pollutants in the air and on surfaces.

### SUMMARY OF THE INVENTION

Thus, it is a first aspect of the present invention to provide an air treatment method and system which is arranged for removing gas-phase pollutants, and/or for disinfecting/sterilizing the air and surfaces in a room.

It is a second aspect of the present invention to provide a portable air treatment system which is arranged for removing high concentrations of pollutants at room temperature.

It is a third aspect of the present invention to provide an air treatment method and system arranged for treating the air in a fast and effective manner, using much less energy for the treatment process compared to the traditional systems and methods.

It is a fourth aspect of the present invention to provide an air treatment method and system that is simple and reliable to use.

It is a fifth aspect of the present invention to provide an air treatment system which does not require addition of expensive oxidizing agents such as hydrogen peroxide, thereby reducing both costs and space for storage facilities.

The novel and unique features whereby these and further aspects are achieved according to the present invention is by providing an air treatment system comprising
a sterilization unit arranged for producing ozone,
a photooxidation unit arranged for subjecting an air flow to a photooxidation process, and
a control unit arranged for in a first operational mode directing an air flow through the sterilization unit, and in a second operational mode directing an air flow through the photooxidation unit.

By providing an air treatment system which can operate in two different modes i.e. in a photooxidation and a sterilization mode, it is possible to specifically remove the undesirable pollutants in the air, e.g. removing either gas-phase pollutants or disinfecting/sterilizing the air and surfaces in a room simply by selecting either the first or second operational mode.

Furthermore, said system can be used in a number of different locations in which different kinds of pollutants are to be removed, thereby providing a simple and inexpensive system without compromising the effectiveness of the individual treatment.

For instance, if the pollutant is an infectious agents, such as a fungi, bacteria, or a virus then the air treatment system according to the invention may be set to operate in the first operational mode via the control unit, as it in these situations is preferred to be able to disinfect and/or sterilize the air and/or one or more surfaces in a contaminated room.

Alternatively, if the pollutant in the air is one or more organic gas-phase compounds e.g. odors, solves, VOC's etc. the system can be set to operate in the second operational mode where the polluted air is subjected to a photo-oxidation process, i.e. an oxidation process caused by light.

Ozone has long been recognized for its ability to be used for sterilization/disinfection purposes due to its exceptionally oxidative activity. In fact, ozone kills bacteria/vira/mould more rapidly than chlorine, and unlike chlorination, which leaves undesirable chlorinated organic residues; ozone leaves few, if any, potentially harmful residues.

However, ozone is explosive when concentrated as either a gas or liquid, or when dissolved into solvents, and it is therefore preferred that the ozone is manufactured on site.

Thus, in one embodiment the sterilization unit comprises a silent electric discharge unit, also known as a corona discharge unit, wherein air or oxygen is passed through an intense, high frequency alternating current electric field for producing ozone.

It is however preferred that the sterilization unit comprises at least one first UV light source arranged for emitting a wavelength that will produce ozone, which in a preferred embodiment is a wavelength between 100 nm and 280 nm. Preferably, the at least one UV light source is arranged for emitting an UV radiation with a wavelength of about 172 nm and/or 185 nm, as said wavelengths have proven particularly suited for producing ozone from oxygen in the air.

In a preferred embodiment the at least one first UV light source is arranged for generating an ozone concentration of between 1 and 300 ppm, preferably between 5 and 100 ppm and even more preferred between 10 and 50 ppm. In this way the ozone concentration is sufficiently high to sterilize/disinfect both the air and the surfaces in a room.

Within the context of the present invention the term “room/area” encompassed both open and closed spaces, it is however preferred that the room, when the air treatment systems runs in the first operational mode can be sealed off to prevent bystanders from being exposed to the high ozone concentrations.

In a preferred embodiment the sterilization unit further comprises at least one second UV light source arranged for emitting UV-light with a radiation capable of sterilizing air. Preferably the UV light source emits radiation in the vicinity of 254 nm (253.7 nm) which is known to sterilize air. However, other wavelengths such as wavelengths around 172 nm and 222 nm have also proven efficient in inactivating microorganisms including vira, and are therefore also contemplated within the scope of the present invention.

The sterilization unit can thus emit radiation arranged both for disinfecting surfaces, by creating ozone that is emitted into the room, and for sterilizing air either via ozone and/or when the air is subjected to radiation when it passes the at least one second UV light source. Such a sterilization unit is highly advantageously for use in a room which is, or may be, contaminated with an infectious agent, e.g. in a hospital, or an office or area used by different people.

Traditionally, mercury lamps have been used for emitting UV-lights in air treatment systems. However, these lamps have the disadvantage that only a fraction of the radiation is in the desired UV-range. The remainder being in the visible and infrared spectrum. This means that a relatively large part of the energy used by the lamps, are not used for generating UV-light, making said lamps relatively ineffective. Furthermore, for these lamps to function, mercury has to evaporate meaning that the lamps will get very hot. Accordingly their ultra-violet outputs are significantly reduced if they are operated at e.g. room temperature. These drawbacks precludes the use of mercury lamps in some situations, or requires cooling of the air before said air e.g. can be used for air-condition and/or ventilation purposes. Further, mercury lamps, and the mercury used in such lamps, pose a significant environmental hazard, and are accompanied by specialized handling and disposal requirements when the lamp reaches the end of its useful life.

It is accordingly preferred that the at least one first and/or second UV light source is at least one first and/or at least one second excimer lamp, respectively.

Excimer lamps are quasi-monochromatic light sources available over a wide range of wavelengths in the ultraviolet (UV) and vacuum ultraviolet (VUV) spectral regions. The operation of excimer lamps is based on the formation of excited dimers (excimers). These excimer formations are unstable and will disintegrate within nanoseconds, giving up their excitation (binding) energy in the form of photons (radiation) at a characteristic wavelength.

The generated radiation (emitted photons in the UV and VUV range) will upon contact with oxygen e.g. present in the air generate ozone.

In a preferred embodiment according to the present invention, the excimers are produced using the rare gases, i.e. Br₂ (289 nm), Cl₂ (259 nm), Ne₂, I₂ (342 nm), Ar₂ (126 nm), Kr₂ (146 nm),

F₂ (158 nm) and Xe₂ (172 nm), or the rare gas halides (e.g. ArBr (165 nm), ArCl (175 nm), ArF (193 nm), KrI (190 nm), KrBr (207 nm), KrCl (222 nm), KrF (248 nm), XeBr (282 nm), XeI (253 nm), XeCl (308 nm) and XeF (351 nm). However, halogens and mercury halogen mixtures (e.g. HgCl (558 nm), HgBr (502 nm) and HgI (443 nm)) are also contemplated within the scope of the present invention.

The excimers may be produced according to the present invention, by silent electrical discharge where the relevant gas for producing the excimers, e.g. xenon, are placed in a gap between two concentric quarts tubes. This technology is well known and will not be discussed in further details in this application, however one preferred excimer lamp for use in the present invention may be a xenon lamp obtained from USHIO America Inc.

The wavelength of the emitted photons depends on the gas used to provide the excimer, and since only a single gas is used in each excimer lamp, the radiation output by the excimer lamps is restricted to a narrow UV wavelength range.

In one advantageous embodiment, the at least one first excimer lamps is a KrI excimer lamps which provide photons with a wavelength of 185 nm or a Xe₂ excimer lamps which produces photons with a wavelength of 172 nm, i.e. the optimal wavelengths for generating ozone. In a similar manner the at least one second excimer lamp is a XeI excimer lamp which emit a wavelength of about 254 nm which is known to sterilize the air, by inactivate microorganisms and vira.

An advantage of using excimer lamps in the sterilization unit is that excimer lamps only generates little heat, making them highly suitable for use in e.g. domestic rooms or hospital facilities, as cooling is not required before the treated air may be submitted into the surroundings.

In addition, excimer lamps have a long lifetime because the electrodes are not in direct contact with the discharge gases and will thus avoid any corrosion during the discharge process and no contamination of the excimer gas, as is often the situation in conventional mercury lamps leading to a short operating lifetime. Finally, non-toxic materials are used in the excimer lamps and thus inherently, there is no environmental problem.

In order to further improve the sterilizing/disinfection capability of the sterilizing unit, the air passing through said unit may further be subjected to a first air particle filter placed upstream of the at least one first and/or second UV light source, i.e. the first air particle filter is placed before the first and second UV light source(s) seen in the flow direction. The first air particle filter may be any kind of suitable filter device arranged for removing particulate material from the air flow, e.g. particulate air (HEPA) filters and/or Ultra Low Particulate Air (ULPA) that are designed to arrest very fine particles such as microorganisms and vira. However, in a preferred embodiment the first air particle filter is an electrostatic precipitator (ESP), in which the particles will be collected by applying an electric field on the air flow. The electric field will charge the particles, causing said particles to be collected on collecting plates in the ESP, thereby purifying the air. Such ESP systems are well known in the art and will not be discussed in further details in this application.

The photooxidation unit is arranged for subjecting the air to be treated to a photooxidation process in which hydrocarbons e.g. organic acids, alcohols, and aldehydes, primary, secondary and tertiary amines, as well as other VOCs, (including pollutants such as odors, solvents etc.), and BTEX present in the polluted air can be removed. It is therefore preferred that the photooxidation unit comprises at least a third UV light source, which preferably is at least one third excimer lamp.

In combination with natural oxygen, UV light creates highly reactive radicals such as excited oxygen species, e.g. OH, O¹D, O³P, as well as ozone, to be generated from oxygen present in the air, which will proceed to oxidize and eliminate the hydrocarbon contaminants present in the air. Accordingly, the process residuals do not require any additional treatment, as they might in the known systems for treating air. Furthermore, the photooxidation process requires neither addition of reagents to interact with the pollutants (besides the compounds that may be generated in the process, e.g. ozone) nor high temperatures.

As described above, only a single gas is used in each excimer lamp, whereby the radiation output by the excimer lamps is restricted to a narrow UV wavelength range. This allows a perfect match with the absorption spectrum of the pollutants/compounds that are to be removed from the air, i.e. the UV-light source e.g. excimer lamps in the photooxidation unit may be selected in order to match the absorption spectrum of the pollutants in the air to be treated (contaminated air).

Furthermore, UV-light is an energy-saving and environmentally, friendly solution, and ultraviolet radiation is powerful enough to break many covalent bonds. Alone it can degrade PCBs, dioxins, polyaromatic compounds, and BTEXs.

It is preferred that the at least one third excimer lamp emits photons having a wavelength in the range between 126 nm and 240 nm, since photons emitted in this range not only will ensure a substantially complete removal of gas/phase hydrocarbon pollutants, but also that the generation of further pollutants, such as NOx, is prevented.

In one advantageous embodiment, the at least one third UV light source emits a wavelength of about 172 nm, which may be obtained by a xenon excimer lamp. The inventors of the present invention have shown that this wavelength in a very energy efficient way is capable of removing substantially all organic gas/phase compounds e.g. VOC's by means of photolysis, and simultaneously at least to some extend also sterilize the air, by inactivating microorganisms and vira. Furthermore said wavelength will in some degree also produce ozone that will assist in oxidizing organic contaminants present in the air.

However, other wavelengths are also preferred within the scope of the present invention. As an example can be mentioned that wavelengths around 222 nm has proven to be effective in destroying double bonds e.g. C═C and C═O, which may be obtained by a KrCl excimer lamps. A radiation peak around 222 nm will, if humidity is present in air to be treated, also provide a photo-induced production of hydrogen peroxide (H₂O₂). Since hydrogen peroxide is a strong oxidation agent this will further ensure an effective removal of organic pollutants.

In a preferred embodiment the photooxidation process is a UV-O₃ photooxidation process, i.e. the gas stream is subjected to a combination of UV and ozone, preferably simultaneously, and in such an embodiment the at least one third UV light source may be arranged for producing ozone, or the photooxidation unit may comprise at least one fourth UV light source specifically arranged for producing ozone, i.e. the at least one fourth UV light source is arranged for operating in an UV-spectrum which produces ozone, i.e. in a UV-spectrum around 185 nm.

In order to promote the productions of OH-radicals in the photooxidation unit, the photooxidation unit may comprise a water vapor delivery system to increase the relative humidity and/or absolute water content of the air to be treated, preferably to a relative humidity at or above 90%.

When the contaminates in the air (both organic and inorganic) are subjected to radiation from the at least one third and optionally fourth UV light source in the photooxidation unit, a number of microparticles may be formed. Thus, in order to remove these from the treated air stream it may be preferred that the photooxidation unit comprises at least one second air particle filter. Said at least one second air particle filter may be similar to the first air particle filter placed in the sterilization unit, or it may be a different kind of filter. It is however preferred that the second air particle filter is an electrostatic precipitator (ESP). However in contrast to the sterilization unit where the first air particle filter were placed before the UV light source(s), seen in the flow direction, the second air particle filter in the photooxidation unit is placed downstream, i.e. after the UV light source(s) seen in the flow direction.

In a preferred embodiment the photooxidation unit further comprises at least one catalyst. Said catalyst may either be adapted for treating the air as a photocatalyst, or it may be arranged for reducing the concentration of submitted ozone into the surroundings.

If the catalyst is adapted for treating the air, the air treatment unit is preferably arranged as a photocatalytic unit, in which the at least one UV-lamp and photocatalyst is mutually arranged such that the catalyst will be irradiated with UV light. Although various photocatalysts may be used in the photocatalytic unit, titanium dioxide is preferred due to the fact that titanium dioxide is generally accepted as a light, strong, and anti-corrosive compound that, if scratched or damaged, will immediately restore the oxide in the presence of air or water.

Alternatively the catalyst is arranged for reducing the content of ozone in the treated air, e.g. by converting ozone into oxygen. Such catalysts are known in the art, and may e.g. be a substrate with a catalyst material of a type known in the art for ozone decomposition, such as a catalyst including platinum and a base metal. Since ozone is hazardous to humans even at low concentrations, as it causes injury on the respiratory system, this embodiment has the obvious advantage that after the sterilization unit has sterilized /disinfected a room/area the photooxidation unit may decompose any remaining ozone in the air thereby ensuring that the area/room is safe.

A person skilled in the art will understand that there will be some overlap when air is treated in the sterilization unit and the photooxidation unit, i.e. hydrocarbons in the air passing through the sterilization unit will be removed to some extent, and microorganisms will also, to some extent, be inactivated in the photooxidation unit. However neither unit will be able to efficiently remove both hydrocarbons from the air and sterilize/disinfect air/surfaces in a room. Thus, by providing an air treatment system incorporating both units, i.e. the sterilization unit and the photooxidation unit, which can be operated individually, it is possible to provide a system capable of providing both process in a single unit and thereby obtaining both optimal removal/sterilization processes, as well as a simple, small and economical system.

Even though it is preferred that the two units operate alternately, i.e. not at the same time, there may be situations where it is preferred to run the two units simultaneously, e.g. if the air/area to be treated is contaminated with a large number of pollutants that may be more effectively removed by using both units.

The control unit is arranged for controlling the operational mode of the air treatment system according to the invention. This may be by a simple manual operation, e.g. switching between the first and/or second operational mode. However, since ozone, that may be produced by both the sterilization unit and the photooxidation unit, is a hazardous compound, it is preferred that the control unit according to the invention, can be operated either remotely e.g. by means of a remote controller or is arranged for being operated automatically.

In a preferred embodiment the air treatment system comprises at least one sensor arranged for either measuring the pollutant to be removed from the treated air and/or the ozone concentration in the room/area to be treated, and that said at least one sensor is arranged for communicating with the control unit. This has the advantages that the selected operational mode can be maintained until the pollutant has been removed to the desired degree, e.g. that a concentration of the pollutant is below a predefined threshold, and/or until the air and/or surfaces has been sterilized/disinfected.

In one preferred embodiment the at least one sensor is an ozone sensor, and is arranged for measuring the ozone concentration in the room/area when the air treatment system is running in the first or second operational mode. Said ozone sensor may in a preferred embodiment monitor the ozone concentration in the room/area when e.g. the sterilization unit is operating, thereby ensuring that said sterilization process is performed with an ozone concentration that is sufficient for disinfection/sterilization the air and/or the surfaces in said room. As an example can be mentioned that inventors of the present invention has found that maintaining an ozone concentration of between 1 and 300 ppm, preferably between 10 and 50 ppm, may be disinfect a 100 m³ room/area in around 30 min.

The control unit is further advantageously arranged for received information from the ozone sensor and for transmitting an alert if the ozone concentration is different from a predetermined value. Said predetermined value may vary during the sterilization process, and may e.g. be a first predetermined value indicating that the ozone concentration is high enough for sterilizing/disinfection the surfaces in the room/area to be treated, and a second predetermined when the ozone concentration is below an ozone threshold value (<0.1 ppm) where it will be safe to re-enter the room.

Said alert can in one embodiment be a simple visual and/or audible alarm transmitted directly by the at least one sensor or the control unit. However, it might be difficult for an operator to be close enough to the air treatment system to be able to visual and/or audible detect such alarms e.g. if high ozone concentrations are produced. It is therefore preferred that the air treatment system comprises one or more operating units arranged for communicating with the control unit, and preferably also for receiving and processing data/signals relating to the values measured by the at least one sensor in the air treatment system, and for transmitting the alert. This will not only enable an operator to constantly monitor the condition of an air treatment system, but also that an operator can monitor and control several air treatment systems simultaneously, and be alerted centrally, if the ozone concentration is different from the predetermined set value, and/or a treatment cycle has been terminated.

The operating unit can be any kind of device capable of receiving and processing the relevant data, but can in a preferred embodiment be a small electronic device e.g. a tablet or mobile phone; a Programmable Logic Controller (PLC) or a personal computer. In any case, the monitoring system comprises relevant software for handling the data received and for controlling the operation of air treatment system. This gives the operator of the air treatment system the possibility of monitoring the system before, during and after use.

It is further preferred that the air treatment system comprises a timer arranged for operating the sterilization unit and/or the photooxidation unit at a predetermined time period and/or to start at a preset time. This means that the operator does not have the responsibility of e.g. timing the period of generating ozone, measuring whether the surfaces have been disinfected, whether the pollutant has been reduced/removed etc. The control unit simply runs the first or second operational mode for the preset period whereby the air treatment becomes fully automated. Said period may e.g. be depending on the area to be treated etc., and can e.g. be selected based on known references in order to ensure that the pollutant in the room/area is removed.

The different steps of the different treatment cycles, i.e. the sterilization process and photooxidation process, and accordingly also the operation of the timer and sensor may be monitored and logged so that the user at a later time can review how the different steps were carried out. This is especially advantageous as the method according to the invention can be optimized whereby it not only will be more effective but also less expensive.

Even though the sterilization unit and photooxidation unit may be provided as separate parts, it is preferred that said units are integrated, thereby providing an integrated unit. However, in order to operate individually of each other, the sterilization unit and the disinfection unit may each comprise a housing having an air inlet and an air outlet, and a fan arranged for drawing the air through the respective unit. Said fan is preferably arranged near the outlet, i.e. after the UV light sources, air particle filters etc., but may be placed at any location of the unit. The only requirement being that the fans are capable of drawing air through the respective units.

It is furthermore preferred that the air treatment system according to the invention is relatively small, i.e. has a dimension and weight which ensures that the system is portable, i.e. easily can be moved from one location to the next, preferably without the need for any physical aids such a hand truck or the like. In a preferred embodiment the air treatment system has a height between 70 and 80 cm, a width between 50 and 60 cm, a depth between 15 and 25 cm, and a weight between 14 and 20 kg, thereby providing a small system. However the system may also be a large system for installation in a room/factory, and e.g. used for large air flow between 1500-20000 m³/h.

The number of excimer lamps in each unit may vary depending on the intended use. However, in order to provide a small and light weight air treatment system it is preferred that the number of UV light sources in each unit is below 10, preferably below 8 and even more preferred below 5. However, larger numbers such as around 20 excimer lamps is also contemplated within the scope of the present invention.

In an alternative embodiment of the air treatment system according to the invention, the UV-light sources in the two units are LED-lamps (or a combination of LEDs and excimer lamps). In such embodiments the LED-lamps are arranged for emitting the wavelengths disclosed for the excimer lamps. However, since LED-lamps can be manufactured to be small, LED-lamps are particularly suitable for small air treatment systems and/or if a high degree of flexibility to design the air treatment system is desired. For instance, the two air treatment units may be designed to have specific shapes in order to ensure that they can fit into existing installations, e.g. conventional ventilation systems. When the UV-light sources are LED-lamps, the respective units may each comprise a large number of LED-lamps e.g. between 500 and 2000 LED-lamps, such as around 1000 LED-lamps.

The UV light sources are preferably distributed evenly in an area of the respective housing, e.g. in one or more rows, and/or a matrix with a plurality of substantially uniformly distributed and parallel UV light sources.

It is furthermore preferred that when the UV light source is an excimer lamp, said excimer lamp is an elongated tube having a longitudinal axis arranged perpendicular to the flow direction in the sterilization unit and the photooxidation unit, respectively. However, in a different embodiment the longitudinal axis of the excimer lamp, at least in the sterilization unit, is arranged in the flow direction of the air, such that air will flow along the length of the excimer lamp, thereby ensuring that the air flowing through the unit has the longest possible contact time with the UV light source and accordingly the emitted photons.

The present invention also relates to a method of treated polluted air using the air treatment system according to the present invention, said method comprises the steps of:
in a first operational mode directing an air flow through a sterilization unit arranged for producing ozone, and/or
in a second operational mode directing an air flow through a photooxidation unit arranged for subjecting said air flow to a photooxidation process,
and controlling the operational mode of the air treatment system.

This will, as already described above provide an effective removal of the pollutants in the contaminated air. By simply selecting the operational mode of the air treatment system the system will either sterilize/disinfected the air and surfaces, and/or degrade gas-phase organic pollutants in the air.

Even though it is preferred to operate the air treatment system according to the invention in either the first operational mode or the second operational mode, they may in one embodiment be operated simultaneously.

The invention will be explained in greater detail below, describing only exemplary embodiments of the exhaust gas treatment system and method with reference to the sole drawing, in which

### BRIEF DESCRIPTION OF THE DRAWINGS

FIG. 1 schematically shown a preferred embodiment of an air treatment system according to the invention,

### DETAILED DESCRIPTION OF THE INVENTION

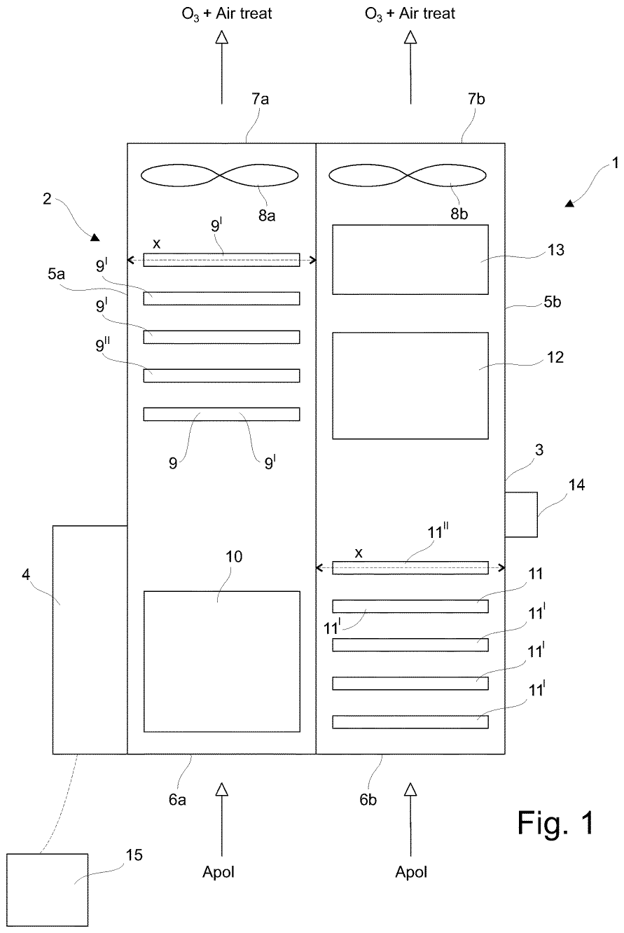

FIG. 1 shows a simplified embodiment of an air treatment system 1 according to the invention. The system 1 is an integrated unit and comprises a sterilization unit 2, a photooxidation unit 3 and a control unit 4.

The sterilization unit 2 and the disinfection unit 3 each comprises a housing 5a,5b having an air inlet 6a,6b, an air outlet 7a,7b, and a fan 8a,8b arranged for drawing the air through the respective unit 2,3. In the embodiment shown the respective fans 8a,8b are arranged near the outlets 7a,7b, but said fans could be placed anywhere in the housing 5a,5b, the only requirement being that the fans are capable of drawing air through the respective units.

The sterilization unit 2 comprises five UV light sources 9 (UV lamps), and a first air particle filter 10. Said filter is preferably an electrostatic precipitator (ESP) as such a filter does not involve large pressure drops etc., whereby large volumes of air can be treated using the air treatment system 1 according to the invention in a fast and effective manner.

In order to optimize the sterilization process, the first air particle filter 10 is placed before the UV lamps 9 seen in the flow direction, thereby ensuring that at least some of the microorganisms have been removed from the air flow Aₚₒₗ that enters the sterilization unit 2 and before said air flow is subjected to the UV radiation when it comes in contact or close proximity to the UV lamps 9.

The five UV lamps 9 may be the same, e.g. arranged for producing ozone, or they may be different i.e. arranged for emitting two or more wavelengths. In the embodiment shown in the figure three of the UV lamps 9′ emits a wavelength of 185 nm, i.e. they will produce ozone, and two of the UV lamps 9″ emit a wavelength of 254 nm, i.e. they will inactivate microorganisms and vira present in the air when said air bypass the UV lamps 9″. Accordingly, both ozone and treated air Aₜᵣₑₐₜ will be emitted from the outlet 7a of the sterilization unit 2.

The photooxidation unit 3 comprises (in addition to the fan 8b), five UV lamps 11, a second air particle filter 12 and a catalyst 13.

As for the sterilization unit 2, the UV lamps 11 in the photooxidation unit 3 may be the same or different. In the embodiment shown four UV lamps 11′ emits a wavelength of about 172 nm, as said wavelength is capable of removing substantially all organic gas-phase compounds e.g. VOC's by means of photolysis. The fifth UV lamp 11″ emits a wavelength of about 185 nm, for producing ozone or about 254 nm for increasing the production of OH radicals, in order to aid in the photooxidation process. If both ozone and OH radicals are desired, the unit may also comprise a sixth UV-lamp for this purpose. Emission of OH radicals has the advantage that less ozone has to be removed after the sterilization step, while maintaining the pollution removal capacity. Thus, even thought the air is treated differently in the two units 2,3, both ozone and treated air will be emitted from both.

When contaminates (both organic and inorganic) in the air Aₚₒₗ entering the photooxidation unit 3 are subjected to radiation by the UV-lamps 11 microparticles may be formed. In order to remove said microparticles, the second air particle filter 12 is placed downstream, i.e. after the UV lamps 11 seen in the flow direction. In order to prevent large pressure drops, said second air particle filter 12 is also an electrostatic precipitator (ESP) as in the sterilization unit.

After the second air particle filter 12, seen in the flow direction, the catalyst 13 is placed. Said catalyst 13 is arranged for converting ozone into oxygen, whereby the ozone generated by the sterilization unit 2 and/or the photooxidation unit 3 effectively can be decomposed.

Adding a catalyst 13 to the photooxidation unit 3 has the obvious advantage, that after the sterilization unit 2 has sterilized/disinfected a room/area the control unit 4 may switch the operational mode to the photooxidation unit 4 where the catalyst effectively decompose any remaining ozone in the air thereby ensuring that the area/room is safe to enter by the operator or other person. Simultaneously, the photooxidation unit will remove/decompose any gas-phase pollutants in the air, if present—thereby providing a very efficient air treatment method.

The UV-lamps 9,11 used in the present invention, i.e. in the sterilization unit 2 and/or the photooxidation unit 3, may be any UV-lamp capable of submitting photons(radiation) with the desired wavelength(s). However, in a preferred embodiment the UV lamps 9,11 are excimer lamps, which offer a number of advantages, high intensity at a defined wavelength, no-self absorption, and flexibility in the construction of the air treatment system according to the present invention. Furthermore, excimer lamps only generate little heat, making them highly suitable for use in domestic facilities, as cooling is not required before the treated air may be submitted into the surroundings. The UV-lamps in the two units 2,3 may however also be LED-lamps and/or conventional mercury lamps, or combinations of excimer lamps, LED-lamps and mercury lamps.

The figure shows the use of five UV-lamps 9,11 in both the sterilization unit 2 and the photooxidation unit 3. However a person skilled in the art will understand that both the sterilization unit 2 and/or the photooxidation unit 3 may contain fewer or more UV lamps, e.g. if a larger UV-emission area is desired or if is desired that the UV-lamps emit several different wavelengths. Accordingly, the system 1 according to the invention can be adapted to be used in both large-area industrial applications and for domestic uses.

The speed of the fans 8a,8b may be adjusted such that the air flow through the respective unit 2,3 can be adapted depending on the area/room to be treated. For instance, the flow rate of the photooxidation unit 3 may be slower than the flow rate of the sterilization unit 2. In this way the UV-light in the photooxidation unit 3 is more likely to get in contact with substantially all contaminates in the air flow passing through said unit, and thereby and effectively clean/treat said flow, as the emitting irradiation will initiate a photooxidation process in the air.

The control unit 4 is arranged for controlling the operational mode of the air treatment system 1 according to the invention. This may be by a simple manual operation, but it is preferred that the control unit is operated remotely or automatically.

In the embodiment shown the air treatment system comprises an ozone sensor 14 arranged for measuring the ozone concentration in the room/area to be treated, thereby ensuring that the sterilization process is performed with an ozone concentration that is sufficient for disinfection/sterilization the air and/or the surfaces in said room.

The control unit 4 is also arranged for received information from the ozone sensor 14 and for transmitting an alert if the ozone concentration is different from a predetermined value. Said predetermined value may vary during the treatment cycles, and may e.g. be a first predetermined value indicating that the ozone concentration is high enough for sterilizing/disinfection the surfaces in the room/area to be treated, and a second predetermined value when the ozone concentration is below an ozone threshold value (<0.1 ppm) where it will be safe to re-enter the room.

Since it may be difficult for an operator to be close enough to the air treatment system 1 to be able to visual and/or audible detect such alert e.g. if high ozone concentrations are produced, the air treatment system also comprises an operating unit 15 arranged for communicating with the control unit 4, and preferably also for receiving and processing data/signals relating to the values measured by the sensor 14, and for transmitting the alert. This will enable an operator to constantly monitor the condition of the air treatment system 1, be alerted centrally, e.g. if the ozone concentration is different from the predetermined set value, notified when a treatment cycle has been terminated etc.

Modifications and combinations of the above principles and designs are foreseen within the scope of the present invention.

1-21. (canceled)

22. An air treatment system for removing at least one pollutant in the air, said system comprising
a sterilization unit arranged for producing ozone,
a photooxidation unit arranged for subjecting an air flow to a photooxidation process, and
a control unit arranged for in a first operational mode directing an air flow through the sterilization unit, and in a second operational mode directing an air flow through the photooxidation unit.

23. The air treatment system according to claim 22, wherein the pollutant in the air is one or more infectious agents and/or one or more organic gas-phase compounds.

24. The air treatment system according to claim 22, wherein the sterilization unit comprises at least one first UV light source arranged for emitting radiation with a wavelength that will produce ozone from oxygen in the air.

25. The air treatment system according to claim 22, wherein the at least one first UV light source is arranged for generating an ozone concentration of between 1 and 300 ppm.

26. The air treatment system according to claim 22, wherein the sterilization unit further comprises at least one second UV light source arranged for emitting UV-light with a radiation capable of sterilizing air.

27. The air treatment system according to claim 22, wherein the at least one first and/or second UV light source is at least one first and/or at least one second excimer lamp.

28. The air treatment system according to claim 27, wherein the at least one first excimer lamps is a KrI excimer lamps which provide photons with a wavelength of 185 nm, and wherein the at least one second excimer lamp is a XeI excimer lamp which emit a wavelength of about 254 nm.

29. The air treatment system according to claim 22, wherein the sterilizing unit comprises an air particle filter placed upstream of the at least one first and/or second UV light source.

30. The air treatment system according to claim 22, wherein the photooxidation unit comprises at least one third UV light source.

31. The air treatment system according to claim 30, wherein the at least one third UV light source is arranged for emitting photons with a wavelength in the range between 126 nm and 240 nm.

32. The air treatment system according to claim 22, wherein the photooxidation unit comprises second air particle filter placed downstream of the at least one third UV light source.

33. The air treatment system according to claim 22, wherein the photooxidation unit further comprises at least one catalyst adapted for either treating the air as a photocatalyst, or for reducing the concentration of ozone.

34. The air treatment system according to claim 22, wherein the air treatment system comprises at least one sensor arranged for either measuring the pollutant to be removed from the treated air and/or the ozone concentration in the room/area to be treated.

35. The air treatment system according to claim 34, wherein the at least one sensor is an ozone sensor arranged for measuring the ozone concentration when the air treatment system is running in the first and/or second operational mode.

36. The air treatment system according to claim 22, wherein the control unit is arranged for received information from the ozone sensor and for transmitting an alert if the ozone concentration is different from a predetermined value.

37. The air treatment system according to claim 22, wherein air treatment system is an integrated unit.

38. The air treatment system according to claim 22, wherein the air treatment system is portable.

39. The air treatment system according to claim 22, wherein the number of UV light sources in each unit is below ten.

40. A method of treating polluted air using the air treatment system according to the present invention, said method comprises the steps of:
in a first operational mode directing an air flow through a sterilization unit arranged for producing ozone, or
in a second operational mode directing an air flow through a photooxidation unit arranged for subjecting said air flow to a photooxidation process,
and controlling the operational mode of the air treatment system.

41. The method of claim 40, wherein the sterilization unit and the photooxidation unit are not operating at the same time.

42. The method of claim 40, wherein the sterilization unit and the photooxidation unit are operating at the same time.
